(19) United States
(12) Patent Application Publication
(10) Pub. No.: US 2011/0015471 A1
(43) Pub. Date: Jan. 20, 2011

(54) PRENATAL AUDIO APPARATUS FOR THE ACOUSTIC STIMULATION OF AN UNBORN CHILD

(76) Inventor: Gabriel Galt, Athens, GA (US)

Correspondence Address: GEORGE R. REARDON, 3356 STATION COURT, LAWRENCVILLE, GA, 30044, US

(21) Appl. No.: 12/503,049

(22) Filed: Jul. 14, 2009

(51) Int. Cl. A61M 21/00; H04R 1/00

(52) U.S. Cl. 600/28; 381/385

(57) ABSTRACT

An audio speaker device is disclosed. The audio speaker is configured for temporary, secure placement directly upon an epidermis surface of a living body. The audio speaker has an output side facing toward the living body. The audio speaker is configured for connectivity to an audio signal source. The audio speaker is configured to communicate an audio signal from the audio signal source to the epidermis surface of the living body in order to transmit the audio signal to within the living body. In at least one embodiment, the audio speaker device is a prenatal audio apparatus for the acoustic stimulation of an unborn child.

### FIELD OF THE INVENTION

The technology described herein relates generally to audio speakers. More specifically, this technology relates to a prenatal audio apparatus for the acoustic stimulation of an unborn child and associated methods for communicating audio to the unborn child within the mother's womb.

### BACKGROUND OF THE INVENTION

The unborn child within the mother's womb is known to respond to external stimuli. As the child develops within the womb, the child becomes more responsive to the external stimuli. By way of example, it is known that the unborn child is able to hear certain sounds while within the womb.

Related patents known in the art include the following: U.S. Pat. No. 4,798,539, issued to Henry et al. on Jan. 17, 1989, discloses a prenatal learning device and method. U.S. Pat. No. 4,934,998, issued to Thomas, Jr. on Jun. 19, 1990, discloses a prenatal audio apparatus. U.S. Pat. No. 5,033,968, issued to Hecht on Jul. 23, 1991, discloses a method and apparatus for safely and intelligibly directing sound to a fetus. U.S. Pat. No. 5,109,421 issued to Fox on Apr. 28, 1992, discloses a fetal speaker system and support belt for maternal wear. U.S. Pat. No. 5,699,558, issued to Min on Dec. 23, 1997, discloses a garment for audio stimulation of the fetus. U.S. Pat. No. 5,873,736, issued to Harrison on Feb. 23, 1999, discloses a prenatal music belt. U.S. Pat. No. 5,898,787, issued to Stanford on Apr. 27, 1999, discloses a fetal and infant audio bag apparatus. U.S. Pat. No. 6,097,822, issued to Min on Aug. 1, 2000, discloses a vest garment for acoustic stimulation of the fetus. U.S. Pat. No. 6,840,775, issued to Sailors on Jan. 11, 2005, discloses a fetal educator strap.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

In various exemplary embodiments, the technology described herein provides a prenatal audio apparatus for the acoustic stimulation of an unborn child and associated methods for communicating audio to the unborn child within the mother's womb.

In one exemplary embodiment, the technology described herein provides an audio speaker device. The audio speaker device includes at least one audio speaker configured for temporary, secure placement directly upon an epidermis surface of a living body. The audio speaker has an output side facing toward the living body. In at least one embodiment, the at least one audio speaker is a pair of stereo audio speakers.

The audio speaker device is configured for connectivity to an audio signal source. The audio speaker is configured to communicate an audio signal from the audio signal source to the epidermis surface of the living body in order to transmit the audio signal to within the living body. The audio signal can include a voice of the mother of the unborn child, music, and the like.

The audio speaker device is configured for placement upon the abdomen area of a human body, for a transmission of an audio signal from the audio speaker to an unborn child within the womb of the human body.

The audio speaker also includes a casing base configured for temporary, secure placement upon a surface of the living body and a casing top configured for operable connectivity with the casing base. The casing base and casing top, joined together, form a housing to house the audio speaker in order to maintain a position of the audio speaker upon the surface of a living body.

The audio speaker device further includes an adhesive pad configured for temporary, secure placement directly upon the epidermis surface of the living body. The adhesive pad has an adhesive on a top surface and a bottom surface of the adhesive pad. The adhesive pad is configured to temporarily secure the at least one audio speaker directly upon the epidermis surface of the living body.

The casing base can further include a speaker grill, through which the audio signal exits the housing.

The audio speaker device still further includes at least one pull tab placed upon one of a top or a bottom surface of the adhesive pad. Each pull tab is configured to cover and seal the adhesive until it is operably pulled to enable use and secure the audio speaker to the epidermis surface of the living body.

In another exemplary embodiment, the technology described herein provides a prenatal audio apparatus for the acoustic stimulation of an unborn child. The prenatal audio apparatus includes a pair of stereo audio speakers configured for temporary, secure placement directly upon an epidermis surface of a living body. Each of the pair of stereo audio speakers has an output side facing toward the living body. The pair of stereo audio speakers is configured for connectivity to an audio signal source. Each audio speaker is configured for placement upon the abdomen area of a human body, for a transmission of an audio signal from the audio speaker to an unborn child within the womb of the human body.

The prenatal audio apparatus also includes a pair of casing bases. Each casing base is configured for temporary, secure placement upon a surface of the living body and a pair of casing tops, each casing top configured for operable connectivity with one casing base. Each casing base and casing top, joined together, form a housing. Each housing is configured to house one of the stereo audio speakers in order to maintain a position of each stereo audio speaker upon the surface of a living body.

The prenatal audio apparatus further includes a pair of adhesive pads configured for temporary, secure placement directly upon the epidermis surface of the living body. The adhesive pads have an adhesive on a top surface and a bottom surface of the adhesive pads. The adhesive pads are each configured to temporarily secure one of the pair of stereo audio speakers directly upon the epidermis surface of the living body.

The prenatal audio apparatus also includes at least one tab placed upon one of a top or a bottom surface of the adhesive pad. Each pull tab is configured to cover and seal the adhesive until operably pulled to enable use and secure the audio speaker to the epidermis surface of the living body.

In yet another exemplary embodiment, the technology described herein provides a method for the communication with and acoustic stimulation of an unborn child. The method includes utilizing a prenatal audio apparatus for the acoustic stimulation of an unborn child, the prenatal audio apparatus having: a pair of stereo audio speakers, the pair of stereo audio speakers configured for temporary, secure placement directly upon an epidermis surface of a living body, the pair of stereo audio speakers each having an output side facing toward the living body; connecting the pair of stereo audio speakers to an audio signal source; and securing the pair of stereo audio speakers upon the abdomen area of a human body, for a transmission of an audio signal from the audio speaker to an unborn child within the womb of the human body.

The method can also include utilizing a housing for each audio speaker, the housing having: a pair of casing bases, each casing base configured for temporary, secure placement upon a surface of the living body; a pair of casing tops, each casing top configured for operable connectivity with one casing base; and wherein each casing base and casing top, joined together, form the housing, each housing to house one of the stereo audio speakers in order to maintain a position of each stereo audio speaker upon the surface of a living body, and placing each audio speaker within one of the housings.

The method can further include utilizing a pair of adhesive pads, the adhesive pads configured for temporary, secure placement directly upon the epidermis surface of the living body. The adhesive pads have an adhesive on a top surface and a bottom surface of the adhesive pads. The adhesive pads are each configured to temporarily secure one of the pair of stereo audio speakers directly upon the epidermis surface of the living body, and securing the pair of stereo audio speakers to the epidermis surface of the living body.

The method can further include utilizing a pair of pull tabs for each adhesive pad, a first pull tab placed upon the top surface and a second pull tab placed upon the bottom surface of the adhesive pad, each pull tab configured to cover and seal the adhesive until operably pulled to enable use and secure the at least one audio speaker to the epidermis surface of the living body; removing each pull tab; securing the top surface of the adhesive pad to and underside of the casing base; and securing the bottom surface of the adhesive pad to the abdomen area of the human body, for a transmission of the audio signal from the audio speaker to the unborn child within the womb of the human body.

The method can further include, wherein the audio signal is a voice of the mother of the unborn child, transmitting the audio signal of the voice of the mother of the unborn child through the pair of stereo audio speakers to the womb.

The method can further include, wherein the audio signal is music, transmitting the audio signal of the music through the pair of stereo audio speakers to the womb.

The method can further include removing the prenatal audio apparatus from the temporary, secure placement directly upon epidermis surface of living body after use and in between uses.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a front perspective view of a prenatal audio apparatus for the acoustic stimulation of an unborn child, illustrating, in particular, application of the apparatus to the abdomen area of the human body and connectivity to the audio output of an audio producing device, according to an embodiment of the technology described herein;

FIG. 2 is a front perspective view of the prenatal audio apparatus depicted in FIG. 1, illustrating, in particular, the casing, speaker wires, and adapter plug, according to an embodiment of the technology described herein;

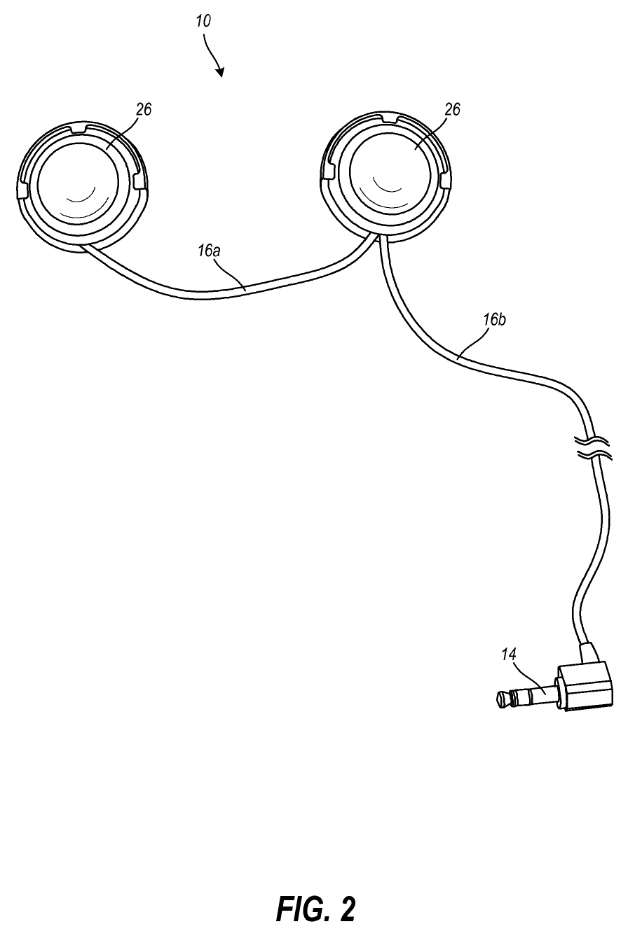

FIG. 3 is a rear perspective view of the prenatal audio apparatus depicted in FIG. 1, illustrating, in particular, the underside of the adhesive gel pad with pull tabs, according to an embodiment of the technology described herein;

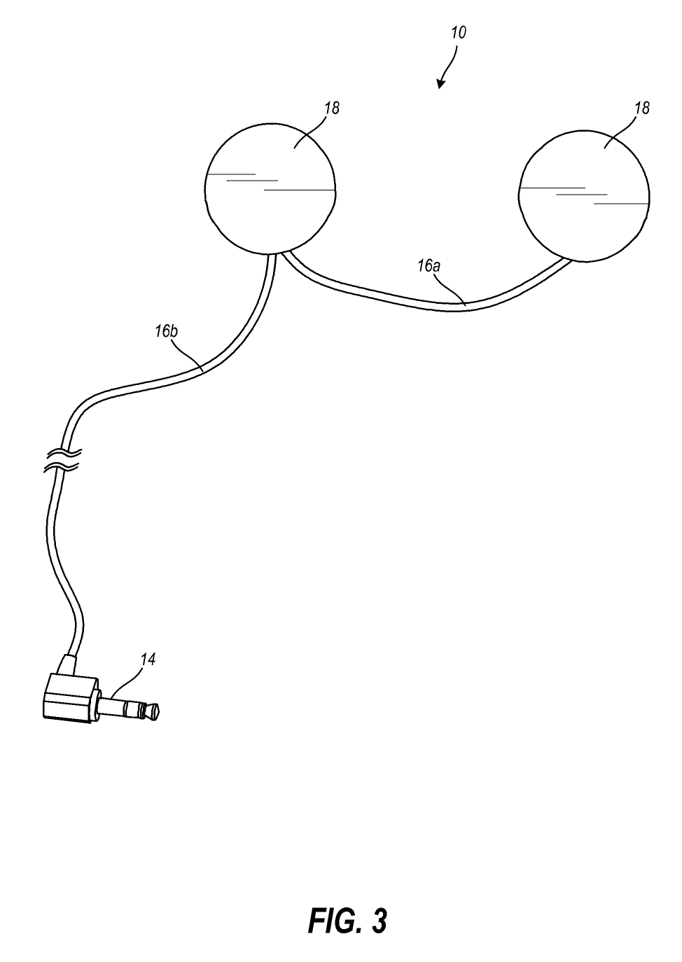

FIG. 4 is an expanded perspective view of the prenatal audio apparatus depicted in FIG. 1, illustrating, in particular, the casing, speaker wires, adapter plug, and adhesive gel pad with pull tabs, according to an embodiment of the technology described herein; and

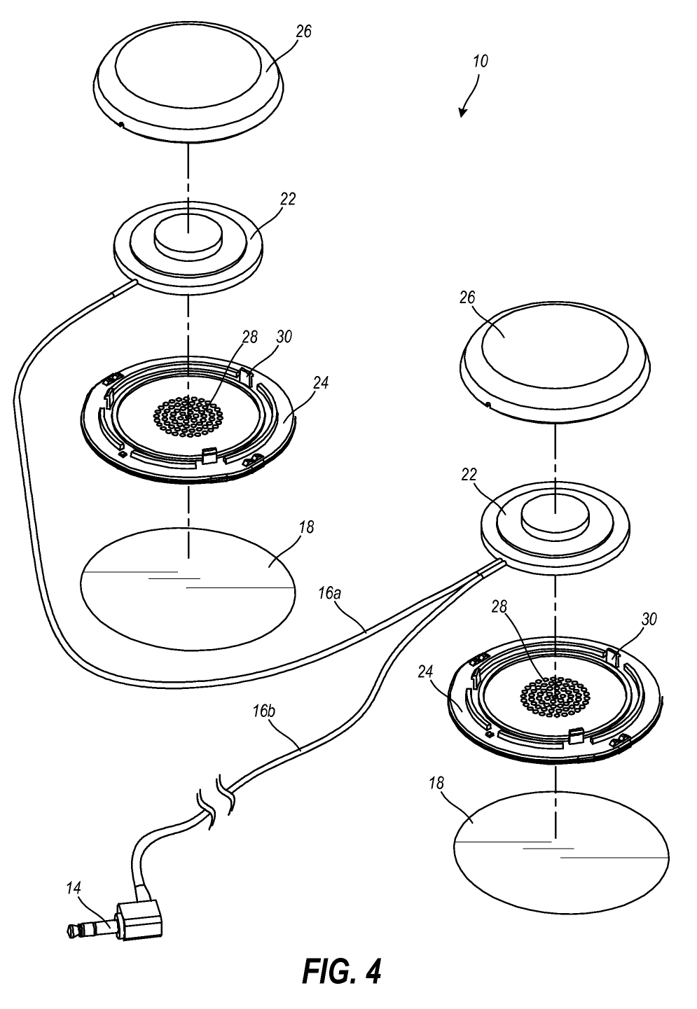

FIG. 5 is a side view of an adhesive gel with pull tabs for use in temporarily adhering the prenatal audio apparatus for the acoustic stimulation of an unborn child to the abdomen area of the human body, according to an embodiment of the technology described herein.

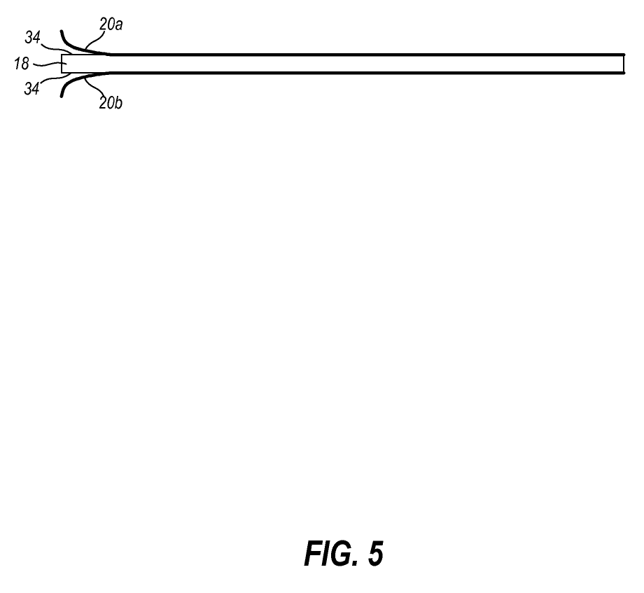

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides a prenatal audio apparatus for the acoustic stimulation of an unborn child and associated methods for communicating audio to the unborn child within the mother's womb.

Referring now to the figures, a prenatal audio apparatus 10 for the acoustic stimulation of an unborn child is shown. The prenatal audio apparatus 10 is configured for connectivity to an audio signal source 32. Connectivity to the audio signal source 32 is attained electrically through wires 16a, 16b, and adapter plug 14.

The audio signal source 32 can provide the voice of the mother of the unborn child, the voice of another, music, speaking, various languages, various sounds, and the like. It will be apparent to those of ordinary skill in the art that alternative audio signals can be communicated from the audio signal source 32 through the prenatal audio apparatus 10.

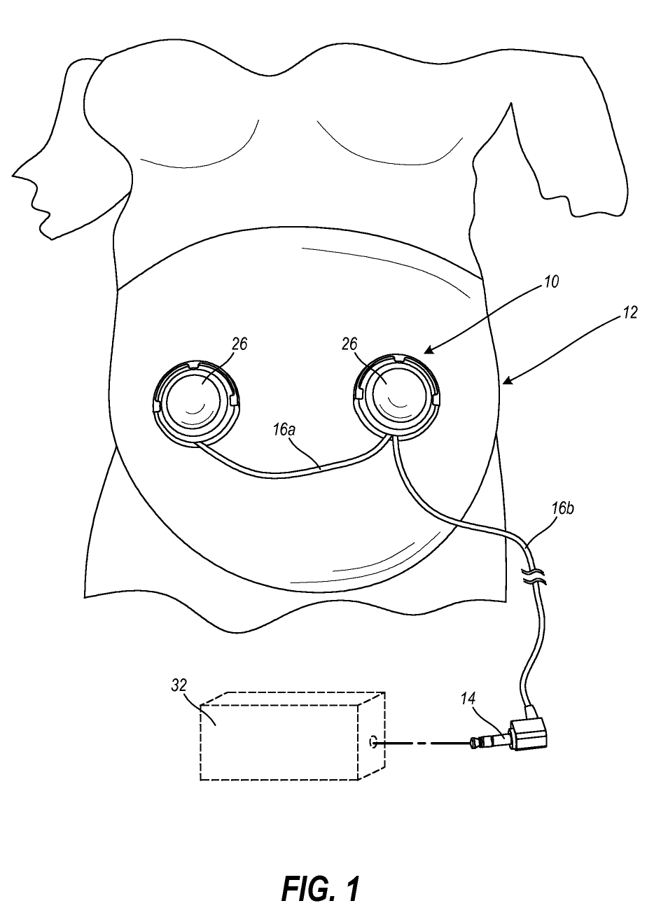

The prenatal audio apparatus 10 is configured for placement upon the abdomen area 12 of a human body, for a transmission of an audio signal from the audio speaker to an unborn child within the womb of the human body. It will be apparent to those of ordinary skill in the art that the prenatal audio apparatus 10 can be placed upon the body in alternative and various locations. The location, an abdomen area 12 depicted in FIG. 1, is shown to illustrate an exemplary location for communicating an audio signal from the audio signal source 32 to through the prenatal audio apparatus 10 to the unborn child within the womb of the mother.

The prenatal audio apparatus 10 is configured for placement upon the abdomen area 12 of a human body without the use of belts, vests, attachments devices, and the like. The prenatal audio apparatus 10 is configured for placement upon the abdomen area 12 of a human body directly with only the use of a minimal adhesive. The adhesive 34 utilized to secure the prenatal audio apparatus 10 to the body is an adhesive known to be safe for temporary application to the epidermis surface of the body.

The prenatal audio apparatus 10 is configured with a pair of stereo audio speakers 22. The pair of stereo audio speakers 22 is configured for temporary, secure placement directly upon an epidermis surface of a living body. The pair of stereo audio speakers 22 each has an output side facing toward the living body when secured to the body. By way of example, when the pair of stereo audio speakers 22 is secured temporarily to the abdomen area 12 of the mother, the speakers 22 are placed in an ideal location to communicate an audio signal from the audio signal source 32 to through the prenatal audio apparatus 10 to the unborn child within the womb of the mother. It will be apparent to one of ordinary skill in the art that the number of speakers 22 can vary from one to many, and that the speakers 22 can be monaural, stereo, or interchangeable between monaural and stereo.

The prenatal audio apparatus 10 is configured with a pair of casing bases 24. Each casing base 24 is configured for temporary, secure placement upon a surface of the living body. As depicted in FIG. 1, placement is upon an abdomen area 12. The prenatal audio apparatus 10 is configured with a pair of casing tops 26. Each casing top 26 is configured for operable connectivity with one casing base 24. Each casing base 24 and each casing top 26, joined together one to another, form a housing. Each speaker housing, formed by the joining of the casing base 24 to the casing top 26, is configured to house one of the stereo audio speakers 22 in order to maintain a position of each stereo audio speaker 22 upon the surface 12 of a living body. By way of example, casing base 24 can be joined to the casing top 26 via a series of latches 30 that lock when twisted or snapped into place. By way of example, casing base 24 can further include a speaker grill 28, through sound can pass.

The prenatal audio apparatus 10 is configured with a pair of adhesive pads 18. The adhesive pads 18 are configured for temporary, secure placement directly upon the epidermis surface of the living body. The adhesive pads 18 are disposable and easily replaceable. Thus, the prenatal audio apparatus 10 is easily reused with a new, sterile set of adhesive pads 18. The adhesive pads 18 have an adhesive on a top surface and a bottom surface and are, in effect, a two-sided adhesive pad. Each adhesive pad 18 is configured to temporarily secure one of the pair of stereo audio speakers 22 directly upon the epidermis surface of the living body.

The prenatal audio apparatus 10 is configured with at least one pull tab 20a or 20b for each adhesive pad 18. By way of example, and when two pull tabs are utilized, a first pull tab 20a is placed upon the top surface of the adhesive pad 18. A second pull tab 20b is placed upon the bottom surface of the adhesive pad 18. Each pull tab 20a, 20b is configured to cover and seal the adhesive 34 until operably pulled to enable use and secure the audio speaker 22 to the epidermis surface of the living body. The adhesive 34 can be an adhesive gel that is known in the art to be safe for direct contact with the epidermis of the human body. In at least one alternative embodiment, the adhesive pad 18 is already secured to a stereo audio speaker 22, and only one pull tab 20b is utilized to remove and then secure the device directly upon the epidermis surface of the living body.

A method is provided for the communication with and acoustic stimulation of an unborn child. The method includes utilizing a prenatal audio apparatus 10 for the acoustic stimulation of an unborn child. The prenatal audio apparatus includes a pair of stereo audio speakers 22. The pair of stereo audio speakers 22 is configured for the temporary, secure placement directly upon an epidermis surface of a living body. The pair of stereo audio speakers 22 each has an output side facing toward the living body. The method further includes connecting the pair of stereo audio speakers 22 to an audio signal source 32. The method further includes securing the pair of stereo audio speakers 22 upon the abdomen area 12 of a human body, for a transmission of an audio signal from the audio speaker 22 to an unborn child within the womb of the human body.

The method can also include utilizing a housing for each audio speaker. The housing includes a pair of casing bases 24. Each casing base 24 is configured for temporary, secure placement upon a surface of the living body. The housing includes a pair of casing tops 26. Each casing top 26 is configured for operable connectivity with one casing base 24. Each casing base 24 and casing top 26, joined together, form the housing, each housing to house one of the stereo audio speakers 22 in order to maintain a position of each stereo audio speaker 22 upon the surface of a living body, and placing each audio speaker within one of the housings.

The method can further include utilizing a pair of adhesive pads 18. The adhesive pads 18 are configured for temporary, secure placement directly upon the epidermis surface of the living body. The adhesive pads 18 have an adhesive 34 on a top surface and a bottom surface of the adhesive pads 18. The adhesive pads 18 are each configured to temporarily secure one of the pair of stereo audio speakers 22 directly upon the epidermis surface of the living body, and securing the pair of stereo audio speakers 22 to the epidermis surface of the living body.

The method can further include utilizing a pair of pull tabs 20 for each adhesive pad, a first pull tab 20a placed upon the top surface and a second pull tab 20b placed upon the bottom surface of the adhesive pad 18. Each pull tab 20a, 20b is configured to cover and seal the adhesive 34 until operably pulled to enable use and secure the at least one audio speaker 33 to the epidermis surface of the living body. The method can further include removing each pull tab 20a, 20b. The method can further include securing the top surface of the adhesive pad 18 to and underside of the casing base 24. The method can further include securing the bottom surface of the adhesive pad 18 to the abdomen area of the human body, for a transmission of the audio signal from the audio speaker 22 to the unborn child within the womb of the human body.

The method can further include transmitting the audio signal of the voice of the mother of the unborn child through the pair of stereo audio speakers to the womb. The method can further include transmitting the audio signal of the music through the pair of stereo audio speakers to the womb.

The method can further include removing the prenatal audio apparatus 10 from the temporary, secure placement directly upon epidermis surface of living body after use and in between uses.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. An audio speaker device comprising:
at least one audio speaker, the audio speaker configured for temporary, secure placement directly upon an epidermis surface of a living body, the audio speaker having an output side facing toward the living body;
wherein the audio speaker is configured for connectivity to an audio signal source; and
wherein the audio speaker is configured to communicate an audio signal from the audio signal source to the epidermis surface of the living body in order to transmit the audio signal to within the living body.

2. The audio speaker device of claim 1, wherein the at least one audio speaker is configured for placement upon the abdomen area of a human body, for a transmission of an audio signal from the audio speaker to an unborn child within the womb of the human body.

3. The audio speaker device of claim 1, wherein the at least one audio speaker comprises a pair of stereo audio speakers.

4. The audio speaker device of claim 1,
a casing base, the casing base configured for temporary, secure placement upon a surface of the living body;
a casing top, the casing top configured for operable connectivity with the casing base; and
wherein the casing base and casing top, joined together, form a housing to house the audio speaker in order to maintain a position of the audio speaker upon the surface of a living body.

5. The audio speaker device of claim 1, further comprising:
an adhesive pad, the adhesive pad configured for temporary, secure placement directly upon the epidermis surface of the living body;
wherein the adhesive pad has an adhesive on a top surface and a bottom surface of the adhesive pad; and
wherein the adhesive pad is configured to temporarily secure the at least one audio speaker directly upon the epidermis surface of the living body.

6. The audio speaker device of claim 4, wherein the casing base further comprises a speaker grill, through which the audio signal exits the housing.

7. The audio speaker device of claim 5, further comprising:
at least one pull tab placed upon one of a top or a bottom surface of the adhesive pad, configured to cover and seal the adhesive until operably pulled to enable use and secure the at least one audio speaker to the epidermis surface of the living body.

8. The audio speaker device of claim 2, wherein the audio signal comprises a voice of the mother of the unborn child.

9. The audio speaker device of claim 2, wherein the audio signal comprises music.

10. A prenatal audio apparatus for the acoustic stimulation of an unborn child, the apparatus comprising:
a pair of stereo audio speakers, the pair of stereo audio speakers configured for temporary, secure placement directly upon an epidermis surface of a living body, the pair of stereo audio speakers each having an output side facing toward the living body;
wherein the pair of stereo audio speakers is configured for connectivity to an audio signal source; and
wherein each audio speaker is configured for placement upon the abdomen area of a human body, for a transmission of an audio signal from the audio speaker to an unborn child within the womb of the human body.

11. The prenatal audio apparatus for the acoustic stimulation of an unborn child of claim 10, further comprising:
a pair of casing bases, each casing base configured for temporary, secure placement upon a surface of the living body;
a pair of casing tops, each casing top configured for operable connectivity with one casing base; and
wherein each casing base and casing top, joined together, form a housing, each housing to house one of the stereo audio speakers in order to maintain a position of each stereo audio speaker upon the surface of a living body.

12. The prenatal audio apparatus for the acoustic stimulation of an unborn child of claim 10, further comprising:
a pair of adhesive pads, the adhesive pads configured for temporary, secure placement directly upon the epidermis surface of the living body;
wherein the adhesive pads have an adhesive on a top surface and a bottom surface of the adhesive pads; and
wherein the adhesive pads are each configured to temporarily secure one of the pair of stereo audio speakers directly upon the epidermis surface of the living body.

13. The prenatal audio apparatus for the acoustic stimulation of an unborn child of claim 12, further comprising:
at least one pull tab placed upon one of a top or a bottom surface of the adhesive pad, configured to cover and seal the adhesive until operably pulled to enable use and secure the at least one audio speaker to the epidermis surface of the living body.

14. A method for the communication with and acoustic stimulation of an unborn child, the method comprising:
utilizing a prenatal audio apparatus for the acoustic stimulation of an unborn child, the prenatal audio apparatus comprising: a pair of stereo audio speakers, the pair of stereo audio speakers configured for temporary, secure placement directly upon an epidermis surface of a living body, the pair of stereo audio speakers each having an output side facing toward the living body;
connecting the pair of stereo audio speakers to an audio signal source; and
securing the pair of stereo audio speakers upon the abdomen area of a human body, for a transmission of an audio signal from the audio speaker to an unborn child within the womb of the human body.

15. The method for the communication with and acoustic stimulation of an unborn child of claim 14, further comprising:
utilizing a housing for each audio speaker, the housing comprising: a pair of casing bases, each casing base configured for temporary, secure placement upon a surface of the living body; a pair of casing tops, each casing top configured for operable connectivity with one casing base; and wherein each casing base and casing top, joined together, form the housing, each housing to house one of the stereo audio speakers in order to maintain a position of each stereo audio speaker upon the surface of a living body; and
placing each audio speaker within one of the housings.

16. The method for the communication with and acoustic stimulation of an unborn child of claim 14, further comprising:
utilizing a pair of adhesive pads, the adhesive pads configured for temporary, secure placement directly upon the epidermis surface of the living body; wherein the adhesive pads have an adhesive on a top surface and a bottom surface of the adhesive pads; and wherein the adhesive pads are each configured to temporarily secure one of the pair of stereo audio speakers directly upon the epidermis surface of the living body;
securing the pair of stereo audio speakers to the epidermis surface of the living body.

17. The method for the communication with and acoustic stimulation of an unborn child of claim 16, further comprising:
utilizing a pair of pull tabs for each adhesive pad, a first pull tab placed upon the top surface and a second pull tab placed upon the bottom surface of the adhesive pad, each pull tab configured to cover and seal the adhesive until operably pulled to enable use and secure the at least one audio speaker to the epidermis surface of the living body;
removing each pull tab;
securing the top surface of the adhesive pad to and underside of the casing base; and
securing the bottom surface of the adhesive pad to the abdomen area of the human body, for a transmission of the audio signal from the audio speaker to the unborn child within the womb of the human body.

18. The method for the communication with and acoustic stimulation of an unborn child of claim 14, wherein the audio signal comprises a voice of the mother of the unborn child, the method further comprising:
transmitting the audio signal of the voice of the mother of the unborn child through the pair of stereo audio speakers to the womb.

19. The method for the communication with and acoustic stimulation of an unborn child of claim 15, wherein the audio signal comprises music, the method further comprising:
transmitting the audio signal of the music through the pair of stereo audio speakers to the womb.

20. The method for the communication with and acoustic stimulation of an unborn child of claim 14, further comprising:
removing the prenatal audio apparatus from the temporary, secure placement directly upon epidermis surface of living body after use and in between uses.
